(19) United States
(12) Patent Application Publication
(10) Pub. No.: US 2006/0252062 A1
(43) Pub. Date: Nov. 9, 2006

(54) System and process for genetic and epigenetic treatment

(76) Inventor: Fred Zaccouto, Paris (FR)

Correspondence Address: SUGHRUE MION, PLLC, 2100 PENNSYLVANIA AVENUE, N.W., SUITE 800, WASHINGTON, DC, 20037, US

(21) Appl. No.: 11/352,299

(22) Filed: Feb. 13, 2006

(30) Foreign Application Priority Data
May 4, 2005 (FR) FR-05/51180
Oct. 7, 2005 (FR) FR-05/53058
Nov. 21, 2005 (FR) FR-05/53533

(51) Int. Cl. C12Q 1/68; C12N 5/06; C12N 15/09

(52) U.S. Cl. 435/6; 435/455; 435/325

(57) ABSTRACT

Transplantation of total or partial organs or tissues from one person to another creates severe immunologic problems and does not obtain a genetic rejuvenation, for example, of half the biological age of the donor's selected tissue. The invention permits, without a return to an embryonic stage, to develop an autologous and genetic rejuvenated (AGR) tissue that can be grafted without graft rejection in the donor's body. In order to reconstitute an organ, every tissue should be treated separately and re-assembled afterwards in a cellular culture. Thus, it becomes possible to repair damaged tissues, such as: retina, myocardium, lung, kidney, hepatic, pancreas, osteoporosis, etc. using their own cells that receive a genetic rejuvenation and that remain adult and immediately functional after their implantation.

This invention concerns systems and processes which treat cells genetically and epigenetically.

Such systems and such processes are useful particularly in the domain of cell treatments, particularly for making autografts from differentiated cells or embryonic or foetal stem cells.

Cell treatments, and in particular autografts, are nowadays performed in order to repair a damaged tissue suffering from a disease, a cell deficiency or necrosis. This technique usually consists of taking a few healthy cells from the tissue concerned, putting these cells in culture for cell multiplication in order to build up a stock or tissue of cells, and to reimplant these cells into the tissue to be treated. These reprogrammed cells can then enable the tissue concerned to recover its original morphological and functional capacities.

For example, this technique is used to repair articular cartilage. Articular cartilage has a limited potential for repair and lesions larger than a certain volume rarely heal well. In order to repair such lesions and to prevent the occurrence of osteoarthritis in patients, chondrocytes immersed in an extracell matrix are taken, the matrix is removed from them for example by enzymatic digestion and they are then put in culture, usually on foetal calf serums or preferably in the patient's serum, and in three-dimensional matrices (for example an agarose, collagen or globin matrix). The removed cells can multiply by mitotic division in this type of culture, then leading to the production of millions of chondrocytes. These chondrocytes can then be reimplanted in the cartilaginous tissue to restore cells and the deficient cartilage.

However, the disadvantage of these multiplication techniques is that removed cells are usually cells that underwent a large number of mitotic divisions. Culture of cells for their multiplication causes a slight reduction of the number of telomeres at each mitosis and this multiplication is often done on cells that are already old, near the end of their life and the end of their functions, and also for which the DNA could be impaired. In particular, it is known that cell aging results in progressive shrinking of telomeres (end of chromosomes). These telomeres condition the remaining number of mitotic divisions. Thus cultivating mother cells on which many mitoses took place can lead to a large colony of aged daughter cells with short survival, and that can also be affected by genic functionality alterations.

The purpose of this invention is to provide genetic and epigenetic treatment systems and processes overcoming the disadvantages such as mentioned above.

In particular, the purpose of this invention is to provide cell treatment systems and processes enabling fast and massive production of healthy cells with improved genic functions and/or capable of being genetically and epigenetically rejuvenated, aged and/or repaired to a desired degree.

Another purpose of this invention is to provide systems and processes for cell treatment leading firstly to reconstitution of an autologous tissue that is missing, failing or that needs to be reinforced or modified, and secondly to genetic rejuvenation of the tissue in which the cells have been implanted.

Therefore, the purpose of this invention is a genetic and epigenetic treatment system for cells to be treated, comprising:
at least one cell to be treated,
a genetic reprogramming medium (GRM) comprising at least natural cytoplasm of at least one genetic reprogramming cell (GRC) and/or synthetic cytoplasm, and
means of bringing at least a part of at least one nucleus of at least one cell to be treated with the said GRM into contact, to modify the biological age and/or repair the said at least one cell to be treated.

Another purpose of this invention is a genetic and epigenetic treatment process for cells to be treated, comprising the following steps:
supply at least one cell to be treated,
supply a genetic reprogramming medium (GRM) comprising at least natural cytoplasm of at least one genetic reprogramming cell (GRC) and/or synthetic cytoplasm, and
bring at least part of at least one nucleus of at least one cell to be treated into contact with the said GRM to modify the biological age and/or to repair the said at least one cell to be treated.

The dependent claims describe various embodiments and applications.

The advantages, characteristics and applications of the invention will become clearer after reading the following detailed description of several embodiments and variants of the invention.

In particular, the invention relates to modification of the environment of a cell nucleus with or without extracell or inter-oocyte transfer, so as to bring the nucleus under the influence of a medium inducing its partial genetic reprogramming, but without causing the nucleus to return until the development of embryonic cells. This medium will cause a better repair of the cell DNA during divisions and aggressions and/or genetic rejuvenation by the action of a medium inverting biological time, such as an oocyte.

In particular, this invention relates to systems and processes applicable to the domain of treatment and/or repair and/or functional and/or morphological cell improvement designed to open up a large number of prospects for the combat against a large number of diseases and also against senescence of tissues very largely due to loss of their functional and morphological capacity for proliferation, regeneration and repair. In particular, the purpose of this invention is a system and processes capable of treating cells of a tissue, particularly for rejuvenating, aging and/or repairing these cells. The cells are then cultivated in an appropriate medium so as to create a stock or tissue of genetically and epigenetically treated cells that can be implanted into the tissue considered or remote from it, where these cells in particular could emit metabolism signalling and/or stimulation proteins and/or peptides, and/or DNA repair enzymes for the tissue considered.

More precisely, systems and processes according to the invention consist of bringing at least part of a nucleus of at least one cell to be treated into contact with a genetic reprogramming medium (GRM).

This GRM comprises at least one natural cytoplasm of at least one genetic reprogramming cell (GRC) and/or synthetic cytoplasm. A reconstituted and/or synthetic cytoplasm may particularly be composed of extracts of embryonic serums, healing serums and/or cells subjected to a metabolic activation. A fully synthetic cytoplasm, for example made by a physical and chemical reconstitution of active substances, is possible. It is also possible to make a GRM in the form of a GRC “broth” with or without nuclei. It is also possible to add extracts of cells or cytoplasm and/or other substances known for their capability to activate nuclear metabolism, such as cells or cell extracts appearing during healing and/or metabolism signalling or stimulation proteins or peptides and/or growth factors and/or cells or extracts of malignant cells. Cells or cytoplasm extracts can be obtained by well known physical or chemical treatments. The advantage of using malignant cells and particularly cytoplasm of malignant cells is due to the fact that metabolic activation, signalling and mitosis factors in them are particularly intense and can temporarily induce a metabolic or nuclear reactivation of a cell to be treated. A cancer contagion is improbable because cancers are usually not transmissible from one tissue to another, and their cytoplasms remain normal.

Cytoplasms from selected malignant cells can be used to temporarily treat nuclei or adult or non-adult cells that are insufficiently capable of dividing spontaneously or in culture, repairing their badly copied DNA, or cells that are functionally failing. It is known that the telomeres in a malignant cell are quickly lengthened, that the malignant cell accelerates and indefinitely prolongs its mitosis, increases repair enzymes of its DNA and increases its auto-, para- and endocrine performances. Therefore, the objective is to selectively transfer a chosen functioning of the malignant cell on the cell to be treated, without risking a teratogenic neoplastic contamination. Since the cancer is not directly contagious for non-malignant cells, even diseased cells, the selective application regenerating malignant cells can for example be done in two ways:
intracell treatment: a nucleus is taken from a malignant cell and is added into the cytoplasm of a cell of the tissue to be treated; this nucleus preferably remains separated from the normal nucleus by a tongue or membrane of a porous biocompatible tissue possibly impregnated with antibodies, the said tissue allowing signalling proteins to pass but preventing genes or chromosomes from passing. Under these conditions, signalling proteins emitted by the malignant nucleus will cause a partial genetic reprogramming of the cell nucleus to be treated, particularly in its failing functions. After one or several mitoses, the malignant nucleus will be removed and the reprogrammed cell can be multiplied in an appropriate cell culture,
extracell treatment: the chosen malignant cells are brought close to a few cells to be treated in a cell culture bath adapted so as to comprise at least one factor capable of increasing the permeability of cell membranes, particularly to signalling proteins. This can be done until observations or genetic, proteomic, biochemical or biophysical tests confirm that a functional reactivation of the nuclear deficiency(ies) to be corrected have been induced. In particular, selective biochips will make it possible to target signalling proteins emitted by the malignant nucleus and selectively reprogram the nucleus to be treated in its required and programmed functions.

In particular, three main types of treatments can be envisaged, namely rejuvenation of the biological age of a cell, aging of the biological age of a cell, and repair of a cell. Rejuvenation of the biological age of a cell also increases the self-repair capacity of this cell, particularly at its DNA.

In the framework of a genetic and epigenetic treatment to rejuvenate a cell, the GRM includes all or part of one or several GRCs. In this case, such a GRC is advantageously an oocyte, an embryonic cell, an embryonic or adult stem cell, a foetal cell or a cell receiving cell recomposed from these cells, or synthesised. Systems, processes and applications to make such a rejuvenation will be described in more detail later.

Aging of a cell is conceivable particularly to treat foetal diseases or newborn diseases, particularly due to embryonic cancers such as glioblastoma. These malignant cells can be reprogrammed by artificially aging them by replacing a malignant nucleus by a healthy nucleus from the same but older autologous or homologous tissue, preferably HLA compatible (Human Lymphocyte Antigen). Thus, the interaction between the older nucleus and the young cytoplasm encourages some temporarily accelerated aging of at least the cytoplasm of the young cell. Aging can then be increased by multiplication of cells in a culture bath, the young cytoplasm causing accelerated mitoses of the older nucleus, inducing shortening of telomeres. Healthy cells can be sorted after culture, to reimplant a healthy tissue to replace the original diseased tissue.

Another possible application consists of repairing a cell, particularly in its chromosomal composition, by treating only partly the nucleus, for example a chromosome. For example, in the framework of a leukaemia, the diseased chromosome and particularly the “Philadelphia” chromosome can be destroyed during the metaphase in which chromosomes are deployed, for example using an ultra-thin laser beam preferably with a diameter equal to or less than 1 micron. An equivalent healthy chromosome is then removed during the metaphase of an equivalent cell from the patient or an HLA compatible donor, and it is implanted in the malignant cell, particularly during its mitosis. In particular, treatment could be envisaged for a large number of cancers, for example glioblastoma, cancer of the breast or the rectum.

Another possible application is to repair only part of the chromosome. Thus, a specific part of a chromosome can be cut, for example the part for which genes are responsible for graft rejection. This can be done using an ultra-thin laser beam. The equivalent part of the equivalent chromosome is then taken from the graft receiver, which can also be done by laser cutting using an ultra-thin laser beam. This part of the chromosome is then reinserted into the original chromosome, which can be done using plasmides or micromanipulations in nanotechnologies. More generally, this type of repair can be considered to repair any deficiency or malfunction of a part of a cell, particularly due to age.

Various embodiments and applications of cell rejuvenation will now be described in more detail.

According to a first aspect of the invention, a differentiated cell is rejuvenated or regenerated by removing its nucleus (with or without its attached cytoplasm) and it is transferred into the GRM, advantageously into a GRC of the oocyte, embryonic or foetal type cell. This nucleus is left in the GRM for a predetermined time and is then removed. According to a first variant, the nucleus is removed before the end of the telophase of the nucleus, in other words the nucleus is extracted from the GRM before it divides into two cells, in other words before the end of its first mitosis. The inventor has observed that this temporary introduction of a nucleus into a GRM, particularly into a GRC, causes fast and important elongation of telomeres, often synonymous with rejuvenation of the chromosomal material. The regenerated nucleus can then be inserted into a differentiated receiving cell (stem or embryonic cell), preferably enucleated preferably autologous, and preferably from an identical tissue, in which mitotic division can continue and can thus lead to the birth of two daughter cells, for which the nucleic material is regenerated. These cells can then be subjected to a multiplication culture and at least millions of cells can be reached sufficiently differentiated so that they can be functionally and morphologically implanted in the original tissue concerned. As a variant, the nucleus can be removed from the GRM after one or several mitoses, then one (or several) rejuvenated nuclei thus obtained is (are) reinserted into a differentiated and preferably autologous receiving cell, preferably in the original cell of the nucleus.

Note that in the framework of this first aspect of the invention, it may be desirable to open or to at least partially remove the membrane from the GRC to prevent any risk of cell division of the GRC. The membrane is necessary for the cell division phenomenon, while the cytoplasm is the preferred location of genetic reprogramming.

According to one advantageous aspect of the invention, the step to remove and transfer the nucleus of the differentiated cell includes removal of the nucleus, but also at least part of the cytoplasm contained in the differentiated cell in order to find some cytoplasmic components in the GRM, particularly in the GRC, that are initially present in the differentiated cell such as the endoplasmic reticulum, the golgi apparatus, ribosomes and/or mitochondria.

According to a second aspect of the invention, bringing at least the nucleus of a differentiated cell into contact with the said GRM can consist of transferring the GRM into a differentiated cell, for example using a pipette or by a transfer caused by a pressure difference. This can be done by creating at least one slit or opening in the membrane of the differentiated cell, and transferring the GRM into the said differentiated cell through the said at least one slit or opening. Advantageously, the said transferred GRM can be separated or removed after a certain predeterminable or observable time period, sufficient to genetically reprogram the nucleus of the differentiated cell. For example, it would be possible to place a GRC and a differentiated cell side by side and to make an opening in the membrane of the GRC and an opening in the membrane of the differentiated cell and then compressing the GRC to at least partially transfer the cytoplasm from the GRC into the differentiated cell. This compression may be achieved by placing a pipette or similar device above the membrane of the cell to be compressed, preferably blocked in contact with a wall and applying an appropriate pressure. This pressure could also be applied using a preferably viscous fluid that can overflow from the pipette without being detached from it. This compression is maintained for the time necessary for genetic reprogramming of the nucleus of the differentiated cell, then compression on the GRC is eliminated with the effect that the cytoplasm of the GRC transferred in the differentiated cell is at least partially sucked into the GRC. Note that the GRM can be removed before or after the first mitosis of the nucleus of the differentiated cell. As a variant, means can be provided to close the differentiated cell with at least part of the GRM remaining included in it.

The example applications described below refer more generally to the first aspect of the invention described above (temporary transfer of a differentiated cell nucleus into a GRM, particularly into a GRC), but it is understood that they could also all be used with the second aspect of the invention described above (transfer of GRM into a differentiated cell). Furthermore, most examples refer to the use of an oocyte, but any GRC and more generally any GRM may be used to implement these examples.

Firstly, it shall be noted that the oocyte used can possibly be an mammalian oocyte. For example a rabbit or sheep oocyte could be used. Oocytes originating from a differentiation induced from embryonic stem cells (OPCE) can also be created in vitro. These OPCEs, for example obtained by cloning, can originate from the graft receiver and the treated nuclei thus become particularly autologous because the cytoplasm of OPCEs only comprises part of its foreign DNA and/or RNA particularly in mitochondria and ribosomes. A nucleus can also be inserted into the oocyte, for example during an initiating, spontaneous or provoked mitosis, or furthermore chromosomes or genes or parts of nuclei to be treated in an embryonic type cell. Embryonic type cells that can be artificially activated by genetic signalling proteins or peptides or by cell activation or regulation can also be used, creating an environment capable of inducing some genetic neighbourhood reprogramming. Thus, removal of the nucleus from the differentiated cell can advantageously be done in anaphase or during telophase depending on the required degree of genetic rejuvenation. Optical means such as a microscope can be used to observe the mitotic period in progress. If a GRC is then used, it then preferably originates from the same tissue, for example a cartilaginous, myocardial tissue, etc., preferably with the nucleus partially removed and cultivable in vitro, in vivo or in situ. This or these cell(s) will preferably be cultivated for multiplication in embryonic tissues sufficiently long in vivo to obtain partial dedifferentiation. Nuclei thus treated can be left either in embryonic type cells to form a graftable tissue in the organism of the nucleus, or extracted from their receiving cells to induce local intra or trans-membrane cell regeneration in a differentiated and preferably autologous and identical tissue. The nucleus or the nuclear part may also be implanted inside a stem cell, preferably an embryonic or foetal type stem cell.

Such partially and selectively dedifferentiated cells can then be introduced into differentiated cells such as chondrocytes, cells with an immune function, endocrinal cells, cardiac cells, cells derived from tissues on which an anti-cancer treatment has been applied, and a cells of islets of Langerhans, cells with the same origin as a graft to be transplanted, hepatocytes, etc., in order to regenerate the corresponding tissue. Such an invention can thus be applied with no limitation to regeneration of any sufficiently differentiated cell such as cardiac, renal, bone, tendon, cartilaginous, cutaneous, dermal, epidermal, pancreatic, hepatic, nerve, prostatic, glandular, hematopoietic, nerve, vascular, retinal, dental, desmodontal, spleen, parathyroidal, suprarenal cells, digestive or respiratory tracts, etc. Starting from a certain degree of dedifferentiation, these cells lose their immunogenic capacity and can sometimes be used to regenerate non-autologous tissues. This function also comprises the capability of these genetically activated cells to act at a distance by secretion, release or induction of genetic signalling peptides and/or proteins particularly by specific biochemical molecules. This trans-membrane and/or trans-humoral genetic activation makes these cells capable of actively and continuously stimulating other deficient senescent cells or to inhibit carcinogenic factors.

The system according to the invention and the cell regeneration processes used preferably comprise four successive stages, namely preparation of nuclear material, genetic reprogramming, multiplication in culture and reimplantation in the organism from the nucleus.

Preparation of the nuclear material consists of removing the nucleus from the sufficiently differentiated cell preferably with more or less cytoplasm in order, if possible, to keep cytoplasmic components such as mitochondria, ribosomes, the endoplasmic reticulum, the Golgi apparatus, lysosomes, peroxisomes, etc., of the initial differentiated cell at the oocyte hosting this removed nucleus. The inventor supposes that this step enables synchronous reprogramming of the various vital structures around the nucleus and probable conservation of the cell “morpho-temporal field”. It is also possible that such a regeneration process has previously taken place on some constituents of nuclear material such as chromosomes, a set of genes, one or several isolated genes (natural, recombined, semi-synthetic or synthetic). In this way, some elements of the preparation will have a different biological age. A segment of vegetal DNA, for example coding for vitamin C, E, folic acid, etc., may also be combined with a gene or a chromosome, for example expressing erythropoietin or various albumins, for example during the metaphase, or to the nuclear membrane in the anaphase, telophase or a corresponding interphase.

The membrane-cytoplasmic receiving cell (oocyte) for treatment of cytoplasmic reprogramming elements may sometime be too small for the cell elements to be treated. Examples include simultaneous treatment of a nucleus with part of its cytoplasm, or several nuclei that are sometimes different such in a nephron, a muscle cell, a myocardial autorhythmic cell, a hair follicle, an epidermic melanisation unit, an epidermis-dermis unit, a glandular unit, a hepatobiliary unit, a retinal functional unit (such as a pigmented epithelium—cones, rods, bipolar cells, horizontal cells and Muller cells), a vascular unit (endothelial and myoarterial cell), a hematopoietic unit, a neuro-glio-dendritic unit, an ovarian unit of Graaf follicles, etc. An enlarged membrane cytoplasmic receiving cell with a preserved oocyte function (RAF) may be necessary to treat a unit with several nuclei by a regeneration system or a process according to the invention. Such a RAF may be made by bonding the corresponding membranes of several preferably homologous or autologous oocytes of mammals, for example by manual or robotic micromanipulations, preferably preserving each corresponding cytoplasm within its corresponding membrane and creating a spherical, ovoid or cylindrical type volume. Such a manipulation requires protection of the vital environment for each oocyte. For example, this type of membrane binding may be made using a micro laser beam, a small heating light beam, a biological binding, etc.

In vitro multiplication is preceded by the introduction of nucleo-cytoplasmic material into a preferably enucleated cell identical to the cell from which the nucleus originates, and at least with recoverable vitality. For example, with existing multiplication techniques, about half a billion cells can be obtained from a few tens of cells in two weeks. In the present case, the inventor has observed that the regeneration process according to the invention enables fast and important lengthening of telomeres in less than a day, thus counterbalancing their irremediable shortening resulting from such large numbers of successive replications. This multiplication may also be done in vivo but is usually much slower and often requires sufficient in vitro priming. This reduces the quantity of cells necessary and the severe shrinking of telomeres and probably enables a better functional adaptation and a greater genetic influence from a distance.

It may be desirable to regenerate a plurality of cells representing a functional organic unit, for example such as a nephron, pigmentary retinal cells of different categories or alveoli of the lungs to form a genetically rejuvenated organic functional unit. For example, it would be possible to envisage cell micromanipulations to create an enlarged chamber with a genetic and epigenetic reprogramming function capable of inverting in time the evolution of the biological age of nuclei and/or multiple cytoplasms introduced in them. To achieve this, cells with an oocyte function may for example be cut into two parts, preferably by a cold light micro laser beam. These two parts are opened and their membranes may be fixed on a proteic layer such as globin, which was preferably applied on a flexible surface. This lawn of oocyte or embryonic membranes includes cytoplasms near the top. When a sufficient surface area of such a cytoplasmic velvet (VC) is formed, it is possible to place several differentiated cell nuclei on it with or without their cytoplasm and then roll the VC around them as closely as possible. This interactive cell sandwich will preferably remain in the classical nutrient cell culture liquid for the time chosen to obtain the desired mitosis phase. Simultaneous rejuvenation of several nuclei belonging to an organic functional unit can then be obtained that can be multiplied either in the state of isolated cells which requires that the multiplied cells should be rearranged in their functional order, or in the state of a set of cells already placed in their functional order.

The regeneration process according to the invention is particularly suitable for diseases characterised by a cell deficiency or failure (diabetes, myocardial infarction, hepatitis, renal insufficiency, drop in the retinal function or genetic diseases responsible for an immunological deficiency) and for cancers occurring beyond a certain age such as cancers of the prostate, breast and colon.

This cell regeneration is applicable to many types of cells and can therefore create controlled regeneration tissues to heal a large number of organic and tissue lesions. Thus for example, the use of ultrasound guidance with a transrectal or transdermal needle or an endoscopic probe to remove the prostate cells that will be completely or partially treated and for example reimplanting them into the prostate, this induced remote cell rejuvenation, particularly by signalling proteins, can sometimes prevent the development of a local cancer, slow its growth or even destroy all metastases. Thus, an autologous or even homologous ophthalmic retina for which a functional cell unit, for example composed of a few cells of pigmented epithelium, cones, rods, bipolar cells and/or Muller cells, that has been removed and regenerated, can be very useful in cases of AMD. A serious renal insufficiency can be fought by the implantation of partially dedifferentiated cells obtained for example after transfer in and then outside oocytes, of nuclei with different nephron cells. Osteoarthritis can be treated by implantation of chondrocytes originating from cell regeneration. The same is true for cutaneous surfaces and hair follicles, and particularly to regenerate and/or colour whitened hair, for example by transferring one or more nuclei or parts of nuclei of hair follicles, melanocytes and keratinocytes into one or several oocyte(s), for regeneration of the hair and/or its colour. Yet another application of the invention could be to reinforce or recreate thymic functions by genetic rejuvenation of homologous thymic cells or possibly autologous thymic cells sufficiently dedifferentiated to actively reanimate immuno-protective functions of the body.

Different applications of the system and the process according to the invention will now be described in more detail. Not all of the steps necessary for cell regeneration will be repeated in the following, the overall principle remaining the same and being adaptable to each case by those skilled in the art. Remember also that the two aspects of the invention, namely firstly temporary transfer of a differentiated cell nucleus into a GRM (particularly a GRC) and secondly the transfer of GRM into a differentiated cell, can be used.

Thus, it is known that aging leads to renal insufficiency with progressive anaemia, these two factors creating chronic fatigue in persons. The objective here is to encourage renal regeneration, particularly by reconstituting some nephrons and cells producing erythropoietin.

A kidney can be regenerated in vitro from different nephron cells (CNE) obtained for example by surgical or endoscopic renal biopsy under visual control. CNEs will be treated by the treatment according to the invention, for example to reduce their biological age by three quarters, and the CNEs thus obtained will be amplified. At the same time, an entire block of nephrons (BNE) is removed, particularly comprising vascularizations, glomeruli with their capsules, small uriniferous tubules and small urinary collection channels. The BNE will be held in survival by connection of its arteries and main veins to an oxygenated artificial circulation of compatible blood plasma or total blood. A visual observation of this BNE in operation can detect different diseased cell segments to be removed and substituted by identical rejuvenated and geometrically reconstituted cell segments by microsurgery in vitro. After verification of good histological and functional integration of the new cell segments on the BNE, this part of the kidney (possibly a complete kidney) will be reimplanted in the patient, with repair of vascular and urinary connections.

One particularly preferred biomedical application for this cell regeneration process concerns degenerative diseases of articulations (osteoarthritis) in general. Cartilage chondrocytes that often degenerate with age may be removed by biopsy, endoscopy, a local surgical operation or arthroscopy and separated from their surrounding cartilage. Their nucleus can then be subjected to the regeneration process according to the invention. After multiplication, the regenerated cells should preferably be reimplanted in the original articulation close to but not on the surfaces of the mobile articular cavity that resists mechanical loads in order to prevent any ruggednesses forming on the mobile surfaces. Sometimes, a disorder in the indirect blood supply to the chondrocytes, that is done largely by imbibition, must be corrected. It is then possible to envisage a graft of an autologous vascular functional tissue comprising small arteries—arterioles—capillaries—venules and small veins surrounding or penetrating into the peripheral cartilage from the articular cavity, and these vascular functional units can advantageously originate from an autologous cell culture post-regeneration process according to the invention. This implantation of rejuvenated cells may take place in the form of layers of lamella preformed in three dimensions in accordance with the local geometry of the previously measured articular cavity, or by spreading in order to cause durable emission particularly of signalling proteins. Post-regeneration process chondrocytes will progressively form a thicker, smoother and well-lubricated cartilage.

Osteoporosis is a degenerative disease of bone tissue that occurs with age. The best approach to combat this disease is to regenerate autologous osteoblasts (and possibly osteocytes) and to reimplant them, preferably at several levels of the bone. Osteoblasts are preferably multiplied in culture with artificial geometric solicitations, particularly by imposing mechanical stresses, for example using a support frame. This support frame may comprise at least one side free to move for movements in a plane. Advantageously, two sides free to move are used in the culture support frame and/or three-dimensional motor rotations may be used. For example, it is easy to perform spaced biopsies at the neck of the femur, under local anaesthesia, by inserting a trocar through the trochanteric massif of the femur that is close to the skin. Treatment of the local osteoblasts and osteocytes thus collected following the cell regeneration process and reimplantation of these rejuvenated cells, for example through the same transtrochanteric channel, can enable local creation of bone remodelling that then fundamentally reinforces sustentation bone trabeculae in the direction of mechanical stresses on the femoral neck and head upwards and downwards towards the body of the femur. A cell regeneration process equivalent to the femur cell regeneration process may be used at vertebrae most severely affected by osteoporosis due to fractures and crushing, possibly in association with fixing solutions and artificial articulations developed by the inventor in patents U.S. Pat. No. 6,835,207 and U.S. Pat. No. 6,692,495. The main or aggravating cause of osteoporosis is aging and the treatment according to the invention in this case is also a preferred solution. Compression or fractures at the spinal column make walking and leg movements difficult. In this case, samples particularly of some osteoblasts and osteocytes should be taken from the main affected vertebrae, for example by posterior transcutaneous puncture, to submit them to a treatment according to the invention and to reimplant them, preferably in the original vertebra as close as possible to the original location of the cell to be treated, and preferably with preliminary in vitro amplification. The same process can be applied at the long bones.

This invention can also be applied to individuals who have suffered severe inflammation, particularly by reactional weakening of the different lymphocytes producing antibodies and pro- and anti-inflammatory cytokines. The regeneration process according to the invention can then be used to revive the number and function of these lymphocytes. To achieve this, these lymphocytes may be subjected to the process according to the invention by placing a lymphocytic nucleus into an oocyte, possibly but not necessarily in the presence of traces of antigens created by the infection concerned in the oocyte cytoplasm. In the presence of antigen traces created by infection placed in the oocyte cytoplasm, lymphocytes are rejuvenated and multiplied and then reimplanted in the organism where they have already “memorised” dangerous antigens and then produce large quantities of the corresponding antibodies, or have them produced. If antigens are placed in the cytoplasm of the GRC, the presence of specific antigens during the cell regeneration process can “memorise” or exteriorise antigens on cell membranes and optimise the antibody production reaction by their immediate appearance as rejuvenated lymphocytic functions reappear.

This invention is also applicable to the combat against cancer. The treatment according to the invention provides means for creating a customised method of anti-cancer treatment so as to perfect traditional anti-cancer treatments that do not take account of individual biological reactions. The following procedure can be used: samples are taken particularly of hematopoietic, lymphocytic and dendritic cells in the bone marrow or at the periphery, and the different categories are isolated and subjected to the treatment according to the invention. After amplification of these cells in vitro, the cells are cultivated in a nutrient bath close to malignant cells taken from the patient's tumour. It may then be useful to limit nutrients and oxygen in the culture bath so as to stimulate a competitive and survival struggle between the two cell categories. Genetically rejuvenated lymphocytes of the patient will naturally develop specific antibodies against antigens of malignant cells and against some substances and biological factors necessary for metabolisms and secretions of malignant cells. For example, they could be antisense or guide RNA, often small, previously transfected in DNA, particularly lymphocytic, by plasmides carrying selected genes or built for this purpose. If the lymphocytes succeed in destroying the malignant cells, they can be reinjected, preferably after multiplication, into the organism of the patient from which they originate. On the other hand, if the lymphocytic cells fail in the destruction of malignant cells, the lymphocytic cells will need to be reinforced, particularly by the injection of selected plasmides and/or cosmides. For example, these can provide the polymerase DNA or DNA segments comprising synthetic or natural genes producing new antibodies or specific toxic substances against the cells to be combated. This increases the capacity for production of antibodies and/or stimulates metabolism and lymphocytic mitoses, either by selection of preferably highly immunogenic cells such as so-called “memory effector with reinforced anti-tumoral potential” T lymphocytes, or for example by reinforcing the genetic rejuvenation treatment of lymphocytes using the treatment according to the invention.

Since malignant cells are autologous, the differential genotypical and epigenotypical examinations provide means for knowing the small part of the genome of malignant cells that differ from normal autologous cells from the same tissue (PGD). Identification of the PGD among known PGDs of other malignant cells, preferably from the same tissue from other persons, enables classification for therapeutic purposes. However for the same PGD, the genes concerned may produce different mRNA particularly by editing or differential splicing. Therefore, it is necessary to know the biological and biochemical behaviours of the cancer specific PGD of each patient that may even vary partly in reaction to a therapy, for example biological, of the type according to this invention. It will then be possible to attempt to find known mild viruses or bacteria in vitro such as some selected and/or genetically manipulated bacteriophages and colibacilli.

If a foreign adult homologous cytoplasm (CEH) is introduced into an enucleated oocyte, genetic reprogramming is possible at the mitochondrial DNA and ribosomic RNA. This possibility can be used to protect an adult nucleus placed in an active oocyte against subsequent transfections by the oocyte cytoplasm as they produce themselves during conventional cloning. Partial cloning removes the nucleus to be treated (NT) from the oocyte before its first cell division and replaces this reprogrammed nucleus in a preferably enucleated cell identical to its original cell. Thus, the oocyte cytoplasm is remote from the NT nucleus at the time of the division of this nucleus, and this division takes place inside an original cytoplasm (CO) of the nucleus NT. The CO should be genetically reprogrammed and its quantity should be increased. To achieve this, a second oocyte identical to the first can be taken and part of its cytoplasm can be sucked in and replaced by a cytoplasm of a cell identical to the cell of the NT nucleus (CCINT). After a required time, the CCINT from this oocyte is removed and the NT nucleus that has kept some its original cytoplasm CO is surrounded by the reprogrammed and recovered CCINT before the NT nucleus, thus repacketed, is inserted into an original cell of the NT nucleus, preferably enucleated and from which part of its cytoplasm has been removed. If necessary, this cell can be increased in size using one of the previously described membrane manipulations.

This invention can advantageously be applied to ulcers. Chronic ulcers often take a very long time to heal, particularly in the legs, and this healing often leaves severe cutaneous and subcutaneous after effects. Other ulcers never heal. In this case, the regeneration process according to the invention should be used to treat at least one epidermal-dermal functional unit of the patient preferably taken from healthy skin close to the ulcer and, after multiplication, it should be implanted at the location of the ulcer. The implantation can be done directly at the ulcer when there is a sufficient local blood irrigation without serious infection, or otherwise it can be done around the ulcer in a healthy skin region. For example, in order to make such an epidermal-dermal functional unit in simultaneous reprogramming, the GRC(s) in which this unit will be accommodated can be fairly voluminous and therefore it can for example be artificially enlarged using the method described above. At the epidermis, the cell may for example be chosen among a keratinocyte cell, a Langerhans cell, a Merkel cell and/or a melanocyte cell taken alone or in combination, while cutaneous fibroblasts can be taken from the dermis. Epidermis and dermis cells can be placed in distinct oocytes. The epidermal-dermal cells collected after regeneration should be positioned and fixed in the culture bath in a reciprocal conformation similar to that observed naturally whenever possible, so as to encourage functional cell growth and simplify the implantation of the tissue layer regenerated on the receiving skin. During culture of the regenerated cells, it might be possible to rearrange the corresponding position of the different cell categories, or even to cultivate several variant assemblies intended for grafts at distinct locations or with a different morphology or function.

It is also known that DNA copying failures become more important with age, and natural repair mechanisms of these failures become less efficient. In order to avoid this insufficiency, a device according to the invention can take local samples from the injured epidermis and/or the dermis, treat it by regeneration according to the invention and then fabricate an extract of these cells from this genetically regenerated tissue, that for example can be fixed in a cream, solution or similar product for an external cutaneous application. This extract could also be used to create a solution that can be injected using a subcutaneous, intradermal or intraepidermal path. It then becomes possible to quickly and temporarily restore epidermal and/or dermal DNA repair functions. Furthermore in an epidermal application, the invention can genetically combat senescence of the skin by modifying collagens, particularly by rejuvenating them, to restore elasticity to the skin.

Regeneration of zones of necrosed, fibrosed or inactive tissue is another application of this invention. Such injured tissue zones may for example be at a myocardium following an infarction or for example in an organ in which a tumour targeted by a destructive anti-cancer treatment has developed. The invention is also applicable to cardiac valves that may be biological with a limited life (about 10 years). They may also be artificial, with a longer life (about 30 years) but in this case the patient needs to follow very restrictive anticoagulant therapy for life. The invention provides means for creating a cardiac valve with a biological, artificial or mixed substrate, or a substrate repaired by plastics and to coat the surface of this substrate in contact with blood with at least one regenerated autologous cell layer. This coating may be produced from a treatment of cardiovascular endothelial autologous cells taken beforehand by cardiac or vascular catheterism, treated according to this invention and then implanted on the valve. In the case of plastics, this implantation may be done peroperatively, in other words during the operation, by covering at least part of the valve and the valvular ring. In this way, the anticoagulant therapy can become unnecessary.

This invention also provides means for helping with determination of the mechanism responsible for a disorder in the health of a mammalian. The first cause of a disorder to a vital equilibrium is sometimes difficult to find. It is then possible to perform cell regeneration of at least one cell of suspect tissues and if the resultant reprogrammed tissues are different from the normal tissue in its intracell composition or its secretions of proteins and peptides either critically or specifically, the intrinsic causal responsibility of this tissue can be demonstrated. For example, for some diabetics who have suffered from the disease for a long period, it is found that cell regeneration of a Langerhans pancreatic cell, for example removed by endoscope, will have a normal provoked secretion of insulin or glucagon, unlike equivalent cells in which there was no cell regeneration. The origin of pathological conditions appearing after a certain age can be revealed by functional comparison of the existing suspect or found tissue compared with its tissue ancestor now genetically rejuvenated to a determined biological age.

This invention is also useful for diseases characterised by a cell deficiency. In particular, this invention enables regeneration and multiplication of β and α cells of islets of Langerhans that may be reimplanted in the pancreas or elsewhere so as to restore insulin or glucagon secretion in an organism of a patient. The implantation of regenerated hepatocytes can cure hepatic disorders in some cases in which hepatic tissue is destroyed (such as cancers, intoxication or cirrhosis).

Another example application of this invention may be to hold an implant in a bone using an envelope or a simple support structure for regenerated cells. This application includes regeneration of bone cells, and particularly osteoblasts, preferably removed at an early stage of their spontaneous mitosis or provoked in a GRC, and then to place these regenerated osteoblasts before the end of their mitosis in a receiving cell, and preferably an osteoblast cell, cultivate the osteoblasts in an appropriate culture medium in order to obtain an appropriate number and mechanical behaviour, and then distribute the osteoblasts in the form of a sleeve, base, structure or envelope between an artificial bone implant and the bone. The layer of genetically rejuvenated osteoblasts then give good solidification of the bone implant and the bone by osteoblastic growth and thus reinforces the support of the implant in the bone and reinforces the bone structure itself. Furthermore, the use of such regenerated cells enables long term support of the implant in the bone and can present a durable, improved and remedial efficiency better than the different proteic creams usually used based on BMPs (Bone Morphogenic Proteins).

Another possible use of this invention is good histocompatibility between a graft of a donor and the immune system of a receiver. To achieve this, a healthy cell may be removed from the organ of the receiver to be grafted, regenerated using the process described and then transferred into an appropriate receiving cell so as to generate proliferation of these cells. These cells can then be placed around the donor's graft so that the immune system of the receiver recognises critical molecules carried to the surface of the graft as self molecules and thus does not generate a strong immune reaction in the presence of the graft. In particular, histocompatibility can be created as follows:

1) Exchange of chromosomes or chromosome segments by genetic micromanipulation during a chosen phase of the mitosis, which is difficult at the moment because their micromanipulation is not yet sufficiently precise; nanomechanics can currently be used to produce chromosome scale instruments for example to perform punctures, grafts, suctions, transfers, cuts and rotations; progress at this level is expected and possible in the near future.

2) Selective destruction of a chromosome segment carrying genes responsible for tissular incompatibility, for example during a mitosis phase or interphase, for example using a micro laser beam with a diameter equal to or less than 1 micron surrounded by a cylinder of wider rays of visible light in order to guide the laser beam by simple microscopic optical control, that can advantageously be robot controlled.

This invention is also particularly useful in the field of dental stomatology. Missing teeth often have to be replaced by metallic, ceramic or plastic implants, etc. These implants require a sufficiently strong maxillary bone support base to solidly fix the implant. If the volume or quality of the solicited region of the maxillary is insufficient, it is advantageous to remove some cells from this bone location, for example by mouth, to submit them to cell regeneration and appropriate multiplication so as to have a small local bone graft that not only provides a solid bone base but which may for example progressively reinforce the entire maxillary arcade by means of signalling proteins, local cytokines, cell activity regulation molecules and genetic expression regulation molecules. Advantageously, this bone regeneration of the maxillary bone that can be done by local injections of regenerated cells within, in contact with or close to the bone, can be combined with a coating of the implant with at least one layer of regenerated bone and/or desmodontal cells, which will improve fixation, the viscoelastic behaviour and the corresponding solidity of the implant in the bone, and the solidity of the bone itself.

Another example application of the cell regeneration process according to the invention relates to fractures and bone surgery. Some bone fractures and malformations require a surgical operation sometimes making it necessary to have an additional graftable and solid bone mass. This can be obtained by genetic rejuvenation of local cells with multiplication every time that final surgery can be delayed by at least two weeks. This is the case particularly for operations for pseudo-osteoarthritis, vertebral bone deformations in children or degenerative deformations, rheumatoid arthritis or osteoarthritis. In vitro cell multiplication of osteoblast cells should preferably be done taking account of mechanical stresses that they have to resist starting at the culture stage, for example after their implantation in the femur, maxillary, vertebrae, etc. In practice, it is necessary to organise this cell culture that is physiologically confluent if possible in an appropriate nutrient bath, but within a support frame that has at least one side free to move in one plane, or in two planes simultaneously, which makes motor rotation possible. Movements and mechanical forces periodically imposed on the growing tissue in its adapted nutrient solution shall have a gradually increasing amplitude, suddenness and strength, but always in the same main orientation so as to cause mineral and trabicular structures in the right direction. The lines of forces and mechanical strength of these structures in one or several directions correspond to the forces, shock absorbing and viscoelasticities that the regenerated bone tissues should resist after its implantation. The inventor has developed an original adjustable vertebral fixator (U.S. Pat. No. 6,835,207) and an original adjustable vertebral disk (U.S. Pat. No. 6,692,495) both of which can advantageously be combined with vertebral tissue originating from such cell regeneration and for example be used as a base for pedicular attachment screws or for filling compacted or fractured vertebrae or to act as a support structure.

Another application of the invention relates to non-autologous grafts. A major problem relates to rejection of grafts by the receiver. It is known that foetal or near embryonic cells are less rejected. Sufficiently rejuvenated cells, for example by several successive treatments according to the invention, can attenuate the problem of rejects of non-autologous grafts.

Note that the modification of the biological age of a cell (rejuvenation or aging) may be measured by different processes. Thus, times or speeds spent by a cell to recover its membrane potential and its action potential after having been subjected to a constraint (such as lack of oxygen or excess potassium) may be compared before and after treatment. If the recuperation time is shorter, then the cell is functionally rejuvenated. Other processes consist of comparing mitosis repetition rates or mitoses themselves, healing rates or modifications to telomere dimensions (volume) before and after treatment.

Note also that the process according to one aspect of the invention denoted by the term “partial cloning”, is distinct from classical cloning. In classical cloning, the nucleus of a cell is transferred in an oocyte, it will divide and become capable of reproducing the original tissue of the said nucleus in utero or artificially in vitro. This means that the nucleus is in contact with a cytoplasm containing mitochondria and ribosomes that could affect the function and/or evolution of the nucleus, particularly by oocyte's, and therefore foreign, DNA and/or RNA contained in them. This means that unless the oocyte originates from the mother of the original cell quickly after giving birth, classical cloning does not produce purely autologous cells and tissues. Furthermore, even if the oocyte originates from the mother, it is possible that it has different characteristics particularly due to the influence of the environment, therapies, diseases, age, etc. Therefore to obtain a purely autologous tissue, it would be necessary to use an oocyte of the mother obtained at the time of the original birth, but this is rarely possible.

On the other hand, in partial cloning that consists of provisionally introducing a nucleus in an oocyte and then retransferring it into an autologous receiving cell identical to its original cell, the tissue obtained is purely autologous, regardless of the oocyte (or equivalent GRC or GRM cell) used. This means that an oocyte of a mammalian that is not necessarily identical can be used, while guaranteeing maximum genetic purity.

Processes according to the invention can also be used to obtain embryonic cells from an adult nucleus. This is done by surrounding the previously reprogrammed nucleus preferably with additional autologous cytoplasm at the nucleus as described above. The treated nucleus thus packeted is then replaced in an oocyte, which is if necessary enlarged according to the invention, and is left to develop embryonic cell divisions.

Although this invention has been described with reference to various aspects of it, and various application examples it is obvious that it is not limited to this description, since the scope of the invention is defined by the attached claims.

1. Genetic and epigenetic treatment process for cells to be treated, characterised in that it comprises the following steps:
supply at least one cell to be treated,
supply a genetic reprogramming medium (GRM) comprising at least natural cytoplasm of at least one genetic reprogramming cell (GRC) and/or synthetic cytoplasm, and
bring at least part of at least one nucleus of at least one cell to be treated into contact with the said GRM to modify the biological age and/or to repair the said at least one cell to be treated.

2. Process according to claim 1, also comprising the following steps:
separate at least the said part of at least a nucleus of the cell to be treated from the said GRM,
introduce at least the said part of at least one nucleus of the cell to be treated and thus separated into a receiving cell, preferably from which the nucleus has been removed, and preferably autologous, the said receiving cell then being capable of multiplying.

3. Process according to claim 2, in which the said multiplication of autologous cells forms a purely autologous tissue without interference of foreign DNA and/or RNA, particularly mitochondrial and/or ribosomic originating from the said GRM.

4. Process according to claim 2, in which the said step to put at least part of at least one nucleus of at least one cell to be treated in contact with the said GRM includes taking and transferring at least the said part of the said at least one nucleus from the said at least one cell to be treated into the said GRM.

5. Process according to claim 2, in which the said step to separate at least the nucleus of the cell to be treated from the said GRM is done before the end of the first mitosis of the said nucleus.

6. Process according to claim 2, in which the said step to separate at least the nucleus of the cell to be treated from the said GRM is done after one or several mitoses of the said nucleus.

7. Process according to claim 2, in which the said at least part of at least one nucleus of at least one cell to be treated comprises at least one chromosome.

8. Process according to claim 2, in which the biological age of the said at least cell to be treated is modified.

9. Process according to claim 8, in which the said at least one cell to be treated is rejuvenated.

10. Process according to claim 9, in which the said at least one cell to be treated is rejuvenated several times successively, such that its biological age is brought back to a very young age, particularly foetal or near embryonic.

11. Process according to claim 9, in which after rejuvenation of the biological age of a cell, the rejuvenated nucleus is removed and replaced by a nucleus of a non-rejuvenated original tissue, such that the said non-rejuvenated nucleus is in contact with a rejuvenated cytoplasm, interaction between them provoking rejuvenation of the said non-rejuvenated nucleus.

12. Process according to claim 8, in which the said at least one cell to be treated is aged.

13. Process according to claim 12, in which at least part of a nucleus of a young cell is removed, particularly a foetal or embryonic, diseased cell such as a chromosome, and is replaced by at least an equivalent part of an older healthy nucleus from the same tissue, preferably HLA compatible, interaction between the young cytoplasm and at least the part of the older nucleus facilitating a temporary acceleration of aging of at least the cytoplasm of the young cell.

14. Process according to claim 13, in which aging is done by multiplication of cells in a cell culture.

15. Process according to claim 13, in which at least a part of a diseased chromosome is destroyed during the deployment of chromosomes in metaphase, at least an equivalent part of an equivalent chromosome in a older healthy cell, preferably HLA compatible, being removed and implanted in the diseased cell.

16. Process according to claim 15, in which at least a part of a diseased chromosome is destroyed using an ultra thin laser beam, preferably with a diameter equal to or less than 1 micron.

17. Process according to claim 8, in which the modification of the biological age of a cell can be measured by comparing the speed before and after treatment at which a cell recovers its membrane potential and its action potential after having been subjected to a constraint such as lack of oxygen or excess potassium, and/or by comparing the repetition speed of mitoses or mitoses themselves before and after treatment, and/or by comparing the healing rate before and after treatment, and/or by comparing the volume of telomeres before and after treatment.

18. Process according to claim 2, in which the said at least one cell to be treated is repaired, particularly in its chromosomic composition.

19. Process according to claim 18, in which a part of a chromosome to be treated is removed, particularly using an ultra thin laser beam, then an equivalent healthy part of an equivalent chromosome is removed, particularly by laser cutting, and reinserted into the chromosome to be treated, particularly by means of plasmides or micromanipulations, to repair the said chromosome to be treated.

20. Process according to claim 19, in which the said removed part of the chromosome to be treated is the part for which the genes are responsible for rejection of non-autologous grafts.

21. Process according to claim 2, in which the said GRM also comprises substances capable of activating the nuclear metabolism, such as cells or extracts of cells appearing during healing and/or signalling or metabolism stimulation proteins or peptides, and/or growth factors and/or malignant cells or extracts of malignant cells.

22. Process according to claim 2, in which at least a cell to be treated is submitted to a temporary treatment by selected malignant cells, by intracell or extracell path.

23. Process according to claim 2, comprising the following steps:
temporarily transferring the cytoplasm of a cell identical to the cell to be treated in a GRM,
separating the said transferred cytoplasm from the said GRM after reprogramming,
surrounding the said nucleus of the cell to be treated separated from the said GRM with the said reprogrammed cytoplasm before inserting the assembly in a receiving cell.

24. Cancer treatment process, characterised in that it comprises the following steps:
removal of at least one hematopoietic cell at the bone marrow or its periphery,
supplying a genetic reprogramming medium (GRM) comprising at least natural cytoplasm of at least one genetic reprogramming cell (GRC) and/or synthetic cytoplasm,
putting at least a part of a nucleus of a removed hematopoietic cell into contact with the said GRM,
separating at least part of a nucleus of a removed hematopoietic cell from the said GRM,
introducing at least the said part of a nucleus of a hematopoietic cell thus separated in an hematopoietic receiving cell, preferably from which its nucleus has been removed, preferably autologous, to rejuvenate the said hematopoietic receiving cell,
multiplying the said rejuvenated receiving cell in vitro,
reinjecting the said rejuvenated cells to combat cancer.

25. Process according to claim 24, in which the said rejuvenated hematopoietic cells are put into contact in vitro with malignant cells sampled from the tumour, before reinjection, particularly so that the said rejuvenated hematopoietic cells develop specific antibodies against antigens of these malignant cells.

26. Process according to claim 25, in which the said rejuvenated hematopoietic cells are reinforced before reinjection, for example by injection of selected plasmides and/or cosmides to increase the capacity to produce antibodies and/or to stimulate metabolisms and/or mitoses of the said hematopoietic cells and/or by selection of preferably immunogenic cells, such as T lymphocytes, and/or by reinforcing genetic rejuvenation of the said rejuvenated hematopoietic cells.

27. System for genetic and epigenetic treatment of cells to be treated, characterised in that it comprises:
at least one cell to be treated,
a genetic reprogramming medium (GRM) comprising at least natural cytoplasm of at least one genetic reprogramming cell (GRC) and/or synthetic cytoplasm, and
means of bringing at least a part of at least one nucleus of at least one cell to be treated into contact with the said GRM, to modify the biological age and/or repair the said at least one cell to be treated.

28. System according to claim 27, in which the said system is a genetic regeneration system for differentiated cells, particularly for their genetic rejuvenation, the said at least cell to be treated being a differentiated cell, and the said genetic reprogramming medium (GRM) comprising at least the cytoplasm of at least one oocyte, embryonic or foetal cell, and/or synthetic cytoplasm.

29. System according to claim 27, also comprising:
means for separating at least the nucleus of the cell to be treated from the said GRM before the end of its first mitosis,
means of introducing the nucleus of the said cell to be treated thus separated into a receiving cell, preferably from which its nucleus has been removed, preferably autologous, the said receiving cell then being a genetically rejuvenated cell capable of multiplying.

30. System according to claim 29, in which the said means for putting at least the nucleus of a cell to be treated into contact with the said GRM comprises means of removing at least the said nucleus from the said cell to be treated and transferring it into the said GRM.

31. System according to claim 30, in which the said means for removing and transferring at least the said nucleus into the said GRM are adapted for removing and transferring the said nucleus of the cell to be treated with at least a part of its cytoplasm so as to obtain some cytoplasmic components initially present in the differentiated cell, such as endoplasmic reticulum, Golgi apparatus, ribosomes and/or mitochondria, in the GRM.

32. System according to claim 30, in which the nucleus of the cell to be treated and to be transferred into the GRM is removed at an early stage of its first mitosis, such as the prophase, the pro-metaphase or the metaphase.

33. System according to claim 30, in which the nucleus of the cell to be treated and transferred into the GRM is separated from the GRM at the metaphase, anaphase stage or during the telophase.

34. System according to claim 27, in which the said means for putting the at least one nucleus of a cell to be treated into contact with the said GRM comprise means for transferring GRM into the cell to be treated.

35. System according to claim 34, in which the said means for transferring GRM into a cell to be treated comprises means of creating at least a slit or an opening in the membrane of the said cell to be treated, and means such as a pipette for transferring the GRM into the said cell to be treated through the said at least one slit or opening.

36. System according to claim 34, in which the said means of transferring the GRM into a cell to be treated comprise means for at least partially separating the said GRM from the said cell to be treated.

37. System according to claim 34, comprising means of definitively closing off the cell to be treated with at least a part of the GRM remaining in it.

38. System according to claim 36, in which the said means of transferring GRM into a cell to be treated comprise means of opening the membrane of an oocyte, embryonic or foetal cell, means of opening the membrane of a cell to be treated, means of putting the said openings in contact, means of compressing the said oocyte, embryonic or foetal cell to at least partially transfer its cytoplasm into the cell to be treated.

39. System according to claim 38, in which the said means of transferring GRM into a cell to be treated also comprise means of at least partially returning the said transferred cytoplasm into its original cell after a certain predeterminable time, particularly by decompression of the said oocyte, embryonic or foetal cell.

40. System according to claim 27, in which the said GRM comprises at least the extraction of cytoplasm from at least one oocyte, embryonic or foetal cell, the said extract being obtained by treatment of the cytoplasm.

41. System according to claim 27, in which the said GRM comprises at least one genetic reprogramming cell (GRC) that can be an embryonic, oocyte or foetal cell from which the nucleus has preferably been extracted.

42. System according to claim 27, in which the said GRM also comprises substances capable of activating the nuclear metabolism, such as cells or extracts of cells appearing during healing and/or signalling proteins and/or growth and stimulation factors.

43. System according to claim 27, comprising means, for example by endoscopy, of injecting and/or implanting treated cells, capable of distributing the said cells within, close to or far from a tissue to be treated.

44. System according to claim 27, in which the system is adapted to the treatment of cells to be treated, such as cardiac, renal, bone, dental, desmodontal, cartilaginous, pancreatic, hepatic, nerve, prostate, hematopoietic, immune, pulmonary, arterial, retinal, cutaneous, dermal, epidermal, glandular, tendon, vascular, spleen, parathyroid, suprarenal and/or digestive and respiratory tracts.

45. System according to claim 27, in which a plurality of cells representing an organic functional unit are treated together and/or reassembled after isolated genetic treatment to form a genetically treated, particularly rejuvenated organic functional unit.

46. System for genetic regeneration of differentiated cells according to claim 28, comprising:
at least one differentiated cell,
means of cell reprogramming, comprising at least one genetic reprogramming cell (GRC) formed of an oocytes or an embryonic cell,
means of removing the nucleus of the at least one GRC, preferably completely,
means of removing and transferring the nucleus of a differentiated cell into a corresponding GRC,
means of extracting the transferred nucleus of the differentiated cell from the GRC, before the end of its first mitosis,
means of introducing the nucleus of the extracted differentiated cell into a differentiated receiving cell, preferably from which its nucleus has been removed and preferably autologous, the said receiving cell then being a regenerated cell capable of multiplying, the resulting tissue being designed to be positioned within a tissue to be regenerated.

47. System according to claim 46, also comprising:
means of removing at least one determined chromosome from the original nucleus of the GRC extracted from the said GRC,
means of implanting this or these chromosome(s) into the nucleus of the differentiated cell transferred into the said GRC.

48. System according to claim 47, also comprising means of removing from the nucleus of the differentiated cell transferred into the said GRC,.the chromosome(s) corresponding to the chromosome(s) taken from the original nucleus of the GRC.

49. System according to claim 27, comprising means of in vitro cell multiplication of treated cells, comprising a support frame receiving a nutrient bath containing the said cells treated in culture, the said support frame being subjected to movements and/or mechanical forces during the said multiplication.

50. System according to claim 49, in which the said support frame comprises at least one side free to move in one plane, preferably at least two sides free to move in two different planes and particularly capable of making a three-dimensional motor rotation.

51. System according to claim 27, comprising mechanical and/or biological mechanical support means for in vitro growth of treated cells, such as an implant with a very enlarged rough surface adapted for this purpose, the said support means being capable of being positioned in the tissue to be treated, the said treated cells then forming a cell cement between the said tissue to be treated and the said support means.

52. System according to claim 51, in which the said growth support means of treated cells comprise an artificial or biological bone implant.

53. Process for genetic and epigenetic treatment, comprising the use of at least one genetic reprogramming cell and at least one differentiated cell for fabrication of at least one regenerated differentiated cell capable of being reimplanted to treat various pathologies, diseases and/or tissue insufficiencies, comprising the following steps:
supplying at least one genetic reprogramming cell (GRC) formed of an oocyte or an embryonic cell,
supplying at least one differentiated cell,
preferably completely removing the nucleus from the GRC,
removing and transferring the nucleus of a differentiated cell into the GRC,
extracting the nucleus of the differentiated cell transferred into the GRC, before the end of its first mitosis,
introducing the nucleus of the extracted differentiated cell into a differentiated cell, preferably from which its nucleus has been removed and preferably autologous, so as to form a regenerated differentiated cell,
multiplying the said regenerated differentiated cell.

54. Process according to claim 53, comprising the use of at least one genetic reprogramming cell and at least one chondrocyte for the fabrication of regenerated chondrocytes that can be implanted, preferably by endoscopy, transcutaneous puncture under ultrasound guidance or arthroscopy, into a bone or articular cartilage prepared for this purpose, in particular outside its articular surface, to treat an osteoarthritis by regenerating the said cartilage.

55. Process according to claim 53, comprising the use of at least one genetic reprogramming cell and at least one cardiac cell for fabrication of a regenerated cardiac tissue that can be implanted in the myorcardial tissue to regenerate an injured zone following a myocardium infarction.

56. Process according to claim 53, comprising the use of at least one genetic reprogramming cell and at least one healthy cell originating from a tissue to be submitted to an anti-cancer treatment, for fabrication of regenerated healthy cells that can be implanted in the tissue targeted by the anti-cancer treatment to regenerate the said tissue at the end of the anti-cancer treatment.

57. Process according to claim 53, comprising the use of at least one genetic reprogramming cell and at least one β and/or α Langerhans cell for fabrication of regenerated cells that can be implanted in the organism, particularly subcutaneously and/or in the pancreas in order to reconstitute a stock of rejuvenated β and/or α cells with normalised endocrinal function and to increase the quantity of insulin and/or glucagons produced, to regenerate the insulin and/or glucagons secretion function.

58. Process according to claim 53, comprising the use of at least one genetic reprogramming cell and at least one epidermal-dermal unit of rejuvenated autologous tissue, particularly for the fabrication of regenerated functional epidermal-dermal units that can be implanted in ulcerous tissue for the treatment of an ulcer or any cutaneous irreversible lesion or anomaly.

59. Process according to claim 53, comprising the use of at least one genetic reprogramming cell and at least one osteoblast for the fabrication of regenerated osteoblasts that can be implanted in a bone suffering from osteoporosis to regenerate the said bone.

60. Process according to claim 53, comprising the use of at least one genetic reprogramming cell and at least one bone cell for the fabrication of regenerated bone cells that can be implanted in a bone receiving an implant to encourage solidification of the said implant in the said bone, by forming a cell cement during their growth, solidifying the bone and the implant.

61. Process according to claim 53, comprising the use of at least one genetic reprogramming cell and at least one epidermal and/or dermal cell for the fabrication of an extract of regenerated epidermal and/or dermal cells to treat injured epidermis and/or dermis and restore its epidermal and/or dermal DNA repair functions.

62. Process according to claim 61, in which the extract of the cells is fixed in a cream for external cutaneous application.

63. Process according to claim 61, in which the extract of the cells is made in the form of a subcutaneous, intradermal or intra epidermal injectable solution.

64. Process according to claim 53, in which the said in vitro cell multiplication of regenerated cells is made using a support frame receiving a nutrient bath containing the said regenerated cells in culture, the said support frame being subjected to movements and/or mechanical forces during the said multiplication.

65. Process according to claim 64, in which the said support frame comprises at least one side free to move in one plane, preferably at least two sides free to move in two different planes and particularly capable of making a three-dimensional motor rotation.

66. Use of a system and/or a process according to claim 1, to at least partially coat a biological or artificial cardiac valve or cardiac valve repaired by plasty with at least one regenerated autologous cell layer.

67. Use of a system and/or a process according to claim 1, to at least partially regenerate the part of a maxillary bone and the desmodontal tissues that will receive a dental implant.

68. Use of a system and/or a process according to claim 1, to at least partially regenerate the epidermis by regeneration of fibroblasts, creating a modification of the collagens.
